(19) United States
(12) Patent Application Publication
(10) Pub. No.: US 2003/0124174 A1
(43) Pub. Date: Jul. 3, 2003

(54) Method for treating non-neuropathic pain

(75) Inventor: Bradley Stuart Galer, West Chester, PA (US)

Correspondence Address: IP Department, Schnader Harrison Segal & Lewis, 36th Floor, 1600 Market Street, Philadelphia, PA, 19103, US

(73) Assignee: Endo Pharmaceuticals, Inc

(21) Appl. No.: 10/045,341

(22) Filed: Oct. 25, 2001

(51) Int. Cl. A61K 9/70; A61K 31/24

(52) U.S. Cl. 424/449; 514/536

(57) ABSTRACT

A method including topically administering an effective amount of local anesthetic to a patient is disclosed. The method is effective for inducing analgesia for treating non-neuropathic pain. Non-neuropathic pain suitable for treatment according to the invention includes pain associated with sports injuries; sprains; strains; soft-tissue injury; repetitive motion injury; carpal tunnel syndrome; injury to tendons, ligament, and muscles; conditions such as fibromyalgia, bursitis, castrochondritis, myofascial pain, and pain associated with arthritis, inflammation, contusions, post-surgical pain, and nociceptive pain. Preferably, the lidocaine is applied via a transdermal patch applied near the locus of pain.

### FIELD OF INVENTION

The invention relates to methods of treating non-neuropathic pain. Specifically, the invention relates to methods of treating non-neuropathic pain by topically administering a local anesthetic, such as lidocaine, in an effective amount near the pain location. Most specifically, the invention relates to methods of treating non-neuropathic pain by administering a topical lidocaine patch to a patient, where the transdermal drug delivery results in no clinically meaningful serum drug levels nor produces anesthesia at the site of delivery, i.e. analgesia without anesthesia.

### DESCRIPTION OF THE RELATED ART

Pain can be treated with either analgesics or anesthetics. A distinguishing feature of analgesics is that they reduce the perception of pain without causing numbness or complete loss of sensation associated with anesthetics.

Currently, prescription analgesics approved by the Food and Drug Administration (FDA) fall into only two classes of drugs: opioids and anti-inflammatories. Anesthetics fall into a different classification. Opioids work by mimicking the body's natural opioid-like substances, i.e. endorphins and enkaphalins, which are produced by the body to help alleviate pain. These substances, and the opioids, block pain by binding to the opioid receptors found throughout the central and peripheral nervous systems. Anti-inflammatories (including NSAIDs and COX-2 inhibitors) attempt to reduce inflammation produced by the prostaglandin chemical cascade resulting from bodily injury. The FDA recognizes only these two classes as “general analgesics.”

Because of this classification, and the known drugs and their mechanisms of action, it is surprising to learn that a product, traditionally classified as an anesthetic, is useful as a general analgesic.

Pain, as discussed herein, falls into two broad categories: neuropathic pain and non-neuropathic pain. The methods associated with treating one type of pain are not necessarily effective at treating the other.

Neuropathic pain is a particular type of pain that has a complex and variable etiology, distinct from nociceptive or inflammatory pain. It is generally a chronic condition attributable to complete or partial transection of a nerve or trauma to a nerve plexus, whereas non-neuropathic pain, i.e. nociceptive or inflammatory pain, occurs in the setting of a normal undamaged nervous system. Neuropathic pain is characterized by hyperesthesia (enhanced sensitivity to a natural stimulus), hyperalgesia (abnormal sensitivity to pain), allodynia (widespread tenderness, characterized by hypersensitivity to normoxious tactile stimuli), and/or spontaneous burning pain. In humans, neuropathic pain tends to be chronic and may be debilitating.

Non-neuropathic pain is just as complex and variable. Non-neuropathic pain includes common conditions such as arthritis pains, musculoskeletal pains, postoperative pains, and fibromyalgia. Most of these pains, such as arthritis pains, musculoskeletal pains, and postoperative pains, are thought to be caused by damage to soft tissue and bone, resulting in the natural inflammatory response in the face of a normally functioning nervous system. However, some non-neuropathic pains, are less well understood. Conditions, such as fibromyalgia, which lead to non-neuropathic pain despite the belief that the nervous system remains intact and undamaged, are not well understood themselves. Treating such conditions and the associated pain is often difficult due to this lack of understanding. It is an object of this invention to treat this and other non-neuropathic pain.

The invention revolves around the proposition that all pain, neuropathic or otherwise, is transmitted by specialized nerve fibers called “nociceptors.” The normal undamaged nociceptor nerve is only physiologically active and gives a normal discharge (resulting in the perception of pain) when the area of skin it innervates is injured by burn, cut, or bruise. This discharge is a normal function of the nerve. Otherwise, the nerve is silent and no pain is perceived in this region of the body.

However, when the nociceptor peripheral nerve itself is damaged, i.e. neuropathic pain, abnormal sodium channels develop at the site of nerve damage, resulting in (1) ectopic abnormal discharges in the normally silent nociceptor nerve, which causes (2) the development of a pain signal in the nociceptor even though no skin damage has occurred, and hence (3) the perception of abnormal spontaneous neuropathic pain and its accompanying hyperalgesia, hyperasthesia, and allodynia in the skin region it innervates. This is not normal function or discharge. Moreover, because these abnormal sodium channels on the damaged nociceptor nerve have an extremely high affinity for sodium and sodium channel antagonist drugs, extremely low doses of sodium channel blocking drugs delivered by intravenous route, oral route, or topical route can bind to these abnormal sodium channels, reduce the frequency of these abnormal discharges, and thus result in the alleviation of neuropathic pain without the complete blockage of the nerve's transmission and without sensory loss or motor blockade.

However, heretofore, in non-neuropathic pain, because the nervous system, including the nociceptor nerve, is not damaged, it has been believed that these abnormal sodium channels do not develop and the pain is solely a result of the inflammatory process. Until now, treatment of normally firing, undamaged nerves by such low doses of sodium channel blocking drugs has not been used or even contemplated. Non-neuropathic pains have not been treated with very low dose sodium channel blocking agents, delivered by any route. Thus, non-neuropathic pains usually have been treated by NSAIDs and COX-2 drugs, that directly interfere with the inflammatory process. In treating such pains, anesthetics are usually injected directly into either the skin or the nerves in the region. The role of anesthetics in treating non-neuropathic pain results in complete sensory block (numbness) and/or complete motor blockade, thereby stopping the nerve's transmission completely, i.e. analgesia (pain relief) with anesthesia (sensory loss). Clinically, anesthesia is not usually the optimal pain treatment as it renders the patient with a numb body part and, at times, paralysis of the involved body region.

Lidocaine, a well-known topical anesthetic, has been used with success to treat pain associated with nerve injury (i.e. neuropathic pain). Because lidocaine is an anesthetic whose sole mechanism of action is peripheral sodium channel antagonism, its use as an analgesic without anesthesia in treating non-neuropathic pain has, heretofore, gone unexplored. It is surprising and unexpected that such a powerful anesthetic useful in treating neuropathic pain is effective to produce analgesia when treating pain where nerve injury is known not to have occurred. Thus, pathophysiologic events associated with non-neuropathic pain must also, like neuropathic pain, involve the production of high affinity sodium channels in the painful regions' uninjured nociceptor nerves. It can, at this time, only be speculated that the normal release of inflammatory peptides, histamine, or other peptides and chemicals known to occur in non-neuropathic pain injury sites results in the development of high affinity sodium channels on the sites of adjacent nondamaged nociceptor nerves.

Because a great many injuries and pain, if not the majority of occurrences, are not neuropathic in origin, more and better methods of treating non-neuropathic pain are needed. Accordingly, the use of lidocaine as an analgesic without anesthesia in treating non-neuropathic pain can be a useful treatment where traditional analgesics and anesthetics might otherwise be used.

### SUMMARY OF THE INVENTION

A method including topically administering an effective amount of a local anesthetic, such as but not limited to lidocaine, to a patient is disclosed. The method is effective for inducing analgesia without anesthesia for treating non-neuropathic pain. Non-neuropathic pain suitable for treatment according to the invention includes pain associated with sprains; strains; soft-tissue injury (bruises and the like); repetitive motion injury; carpal tunnel syndrome; injury to tendons, ligaments, and/or muscles; conditions such as fibromyalgia, bursitis, castrochondritis, myofascial pain, and pain associated with arthritis, inflammation, contusions, post-surgical pain, and nociceptive pain. Preferably, the local anesthetic, such as lidocaine, is applied via a transdermal patch applied on or adjacent to the locus of pain.

### DETAILED DESCRIPTION OF THE PREFERRED EMBODIMENT

The methods disclosed herein are merely illustrative in nature and are not intended to limit the scope of the invention as set forth in the claims below.

It has been discovered, that a topical local anesthetic drug, such as but not limited to lidocaine, has the ability to relieve pain associated with a wide variety of non-neuropathic pain associated with soft-tissue injury, arthritis, surgical procedures, and conditions such as fibromyalgia. This surprising and unexpected discovery has significance in the clinical setting and in the understanding of pathophysiological pain mechanisms. Other non-limiting examples of topical anesthetics which may be used include benzocaine, prilocaine, lidocaine, dubicaine, mepivacaine, bupivacaine, etc.

The finding strongly suggests that pain associated with these types of injuries and conditions is caused, at least to some degree, by the presence of dysfunctional sodium channels on non-damaged peripheral sensory nerves at the pain locus. Thus, a component of the pain perceived, which is associated with damage to non-nervous system peripheral tissues, is caused by abnormal ectopic nociceptive impulses that are generated by abnormal sodium channels.

It is known that damage to a peripheral sensory nerve produces abnormal ectopic nociceptive impulses and pain, i.e. neuropathic pain. Now, based on the findings above, it is hypothesized that injury to soft tissue results in the release of inflammatory and other chemicals and peptides that also cause the generation of abnormal sodium channels on local, undamaged sensory nerves. This then generates abnormal ectopic nociceptive impulses that result in the sensation/perception of pain at the site of soft-tissue injury.

Because of the generation of these normal nociceptive impulses produced in association with inflammation, the local presence of a sodium channel antagonist drug, such as lidocaine, binds to the abnormal sodium channels and reduces or abolishes the frequency of abnormal ectopic nociceptive impulses, and thereby results in alleviation of non-neuropathic pain. Importantly, and novel to this invention is the alleviation of non-neuropathic pain at the site of injury without the development of anesthesia or skin numbness.

Non-limiting examples of soft-tissue injuries include injury to the tendons, ligaments, muscles or bursa, and sprains and strains, etc. These, and other injuries, if occurring during a participation sport may be referred to as sports injuries. However, it makes no difference how the injury was received. The methods herein are effective in treating a broad range of such injuries. Other types of pain resulting from contusions, inflammation, bursitis, costrochondritis, and myofascial pains may also be treated. Other conditions, such as osteoarthritis, rheumatoid arthritis, fibromyalgia and carpal tunnel syndrome, that result in nociceptive pain can also be treated according to the invention.

Fibromyalgia is a condition that is not easily diagnosed or treated. It is poorly understood with no agreed-upon underlying cause or pathophysiological mechanism. Many authorities believe it is caused by a disorder in the nervous system. Fibromyalgia is often associated with flu-like symptoms, including general body pain, coupled with points of sensitivity (“tender points”) and pain at specific locations on the body. Despite the difficulty of treating this condition, treatment according to the method of the present invention can reduce the sensation/perception of pain associated with the condition without the development of anesthesia at the site of pain alleviation.

According to one embodiment, a transdermal patch containing 5% lidocaine is applied to the skin at or near the locus of pain. The patch may contain other pharmaceutically active ingredients, as is known in the art, or other ingredients to help transdermal migration of the active ingredient, stability of the patch, adhesion and other concerns. Currently preferred is the patch marketed as LIDODERM lidocaine patch, available from Endo Pharmaceuticals, Inc. Varying the size of the patch used varies the dosage. Often a patch is cut and only a portion is used. In some instances, the use of more than one patch may be advisable. Optimal pain relief often occurs when lidocaine patches are applied directly to the skin overlying the entire painful body region.

LIDODERM (lidocaine patch 5%) is comprised of an adhesive material containing 5% lidocaine, which is applied to a non-woven polyester felt backing and covered with a polyethylene terephthalate (PET) film release liner. The release liner is removed prior to application to the skin. The size of the patch is 10 cm×14 cm.

Lidocaine is chemically designated as acetamide, 2-(diethylamino)-N-(2,6-dimethylphenyl), has an octanol:water partition ratio of 43 at pH 7.4, and has the following structure:

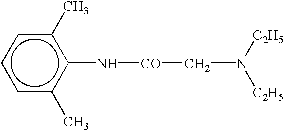

Each adhesive patch contains 700 mg of lidocaine (50 mg per gram adhesive) in an aqueous base. It also contains the following inactive ingredients: dihydroxyaluminum aminoacetate, disodium edetate, gelatin, glycerin, kaolin, methylparaben, polyacrylic acid, polyvinyl alcohol, propylene glycol, propylparaben, sodium carboxymethylcellulose, sodium polyacryslate, D-sorbitol, tartaric acid, and urea.

Another embodiment includes administering a transdermal patch to the patient at the locus of pain where the patch contains approximately 5% lidocaine as the only active ingredient. The remainder of the patch consists of inactive pharmaceutically acceptable agents. Inactive agents do not, in and of themselves, relieve pain. Those skilled in the art will recognize the importance of these inactive agents which facilitate transfer of the lidocaine through the patch and skin, aid in forming the patch itself, or address other concerns and needs. Again, the dosage may be varied by varying the size of the patch.

Administration via transdermal patch is preferred because the application and release of lidocaine can be controlled through known techniques. Although the transdermal patch is preferred, topical application of a composition, including gels, salves, and ointments containing lidocaine will suffice. Topical application of such a composition, in effective amounts, will reduce the sensation/perception of pain in the local area. It should be noted, that when using the current LIDODERM patch delivery system, about 95% of the lidocaine remains unused. Accordingly, the amount of lidocaine or other anesthetic used will vary depending upon the efficiency of the delivery system. Directly applied gels, salves, ointments, etc. may require lesser amounts of the local anesthetic.

Importantly, because the pain associated with many of these injuries and conditions is nearly continuous and can extend over periods of time, the patient benefits greatly from being able to move about and continue with daily activities despite the pain associated with their conditions and injuries. Application of lidocaine as discussed above results in alleviation of pain (analgesia) without numbness or complete loss of sensation (anesthesia) or paralysis. This ability to alleviate pain without numbness or paralysis allows the patient, in many cases, to participate in many daily activities without being burdened by pain or numbness.

Further benefits from topical lidocaine administration are uniformity of treatment between patients since the drug is not subject to absorption through the digestive tract. This also reduces the likelihood of drug interactions and virtually eliminates the possibility of gastrointestinal distress associated with NSAIDs and with opioids. This treatment is particularly effective for strains, sprains, arthritis pains, and post-operative local surgical pain since the analgesic acts locally.

Further benefits include the lack of drug-drug interactions as no clinically meaningful plasma levels develops even with chronic usage.

Further benefits include the lack of the need to titrate the dose, commonly needed with other analgesics, such as NSAIDs, COX-2s, and opioids. Thus an effective dosage is delivered on the first dose. In addition, this may reduce the need for physician visits and phone calls that are associated with titration of medication dosages.

Case Studies

The following case studies are illustrative of the effectiveness of a lidocaine patch in treating various non-neuropathic pains. These are intended only as examples of treatment and are not meant to limit the scope of the claimed invention.

In case studies, many non-neuropathic pains were successfully treated with topical lidocaine patch. From these studies, it is known that topical lidocaine patch results in no clinically meaningful plasma lidocaine levels and no skin anesthesia nor motor block.

Lateral Epicondylitis; “Tennis Elbow”:

A 39 year old male developed lateral epicondylitis (“tennis elbow”) with localized pain and tenderness in the right elbow. The pain was constant and exacerbated by holding any object with his right hand and or any movement of the involved elbow. He placed one topical lidocaine patch (Lidoderm) directly on the skin overlying the painful elbow. Approximately several hours later he noted pain alleviation. He kept a lidocaine patch on his elbow for 3 consecutive days, replacing the patch with a new one every 24 hours, with excellent pain relief and no side effects. There was no appreciable numbness of the skin where the patch was placed. After 3 days of treatment, his pain was completely alleviated and he was able to lift objects and move his elbow joint without pain.

Arthritis:

Case 1

An 89 year old female was suffering with severe osteoarthritis pain of her knees. She was being treated with chronic corticosteroids (oral prednisone) for over 5 years. Initially, the steroids provided good pain relief but the pain gradually had returned over the immediate past year. Nonsteroidal anti-inflammatory drugs were contraindicated due to her prior history of ulcers and the concomitant use of oral corticosteroids. She agreed to a trial of topical lidocaine patch, one patch placed over each knee for 12 hours application per day. After 1 week of treatment, she reported good pain relief in the arthritic knees and no side effects. She stated there was no “numbness” or change of sensation felt under the lidocaine patch.

Case 2

A 59 year old woman with rheumatoid arthritis affecting the elbow. She experiences intermittent severe pain that requires medication. She would rather not take anti-inflammatory medication due to side-effects. She was given lidocaine patch to apply to her painful elbow during these severe pain episodes. She reports a lot of pain relief with no side effects nor skin sensation changes when she applies topical lidocaine patch directly to the arthritic elbow for 24 hours.

Post-Operative Soft Tissue Pain:

A 46-year old male had undergone a surgical repair of a ruptured Achilles tendon. Following 6 weeks in a cast, he experienced moderate to moderately severe pain associated with movement of the surgically repaired Achilles tendon, especially associated with walking and later in the day after nonstrenuous daily activity. In the evening, he applied one patch directly to the skin overlying the Achilles tendon. Within 30-45 minutes he began to experience pain relief. While walking, he reported minimal pain and perceived improved mobility; this exact movement during similar times without the use of the lidocaine patch resulted in moderate to moderately-severe pain and a stiffer gait. Most noticeably, was that pain due to active walking was significantly reduced, but also low grade soreness due to a full day of walking was also significantly reduced. He kept the patch in place while sleeping resulting in minimal sleep interruption due to pain associated with movement during the night.

Ankle Sprain and Cramping Pain:

A 39 year old male suddenly experienced severe cramping pain in his left ankle one evening that prevented him for being able to fall asleep. The pain was so severe he was unable to put any weight on the ankle and had severe pain associated with flexion/extension of the ankle. He applied one topical lidocaine patch to the ankle and began to experience pain relief within 15 minutes. Within 1 hour, his pain was minimal and he was able to fall asleep. He awoke the next morning with no pain and was able to walk on the ankle with no pain. However, approximately 12 hours after patch application, the pain began to gradually return. Another patch was applied for 12 hours and the pain resolved again. When the second patch was removed 12 hours later, his pain was completely resolved and he was able to walk with no pain. No loss of sensation was noted on the skin where the patches were applied.

Poison Oak Pain/Itching:

A 39 year old female was suffering from pain and severe discomfort from itching on her arms due to poison oak. She applied lidocaine patches to the painful and itching skin region. Within 30 minutes she began to report relief of the pain and itching directly underlying the site of patch application. Within 1.5 hours she reported nearly complete pain and itching relief.

Those skilled in the art will appreciate other variations and improvements on the methods disclosed and claimed herein. All such obvious variants are considered within the spirit and scope of the claims below.

What is claimed is:

1. A method for treating non-neuropathic pain comprising topically administering a composition containing a local anesthetic to a patient near a pain locus in an amount sufficient to produce analgesia without causing anesthesia.

2. The method of claim 1 wherein said local anesthetic is lidocaine.

3. The method of claim 1 wherein said local anesthetic is applied from a transdermal patch.

4. The method of claim 3 wherein said patch comprises 1-10% local anesthetic.

5. The method of claim 3 wherein said patch comprises 11-10% lidocaine.

6. The method of claim 3 wherein said patch comprises 4-6% lidocaine.

7. The method of claim 2 wherein said local anesthetic is applied from a transdermal patch comprising 5% lidocaine.

8. The method of claim 1 wherein said non-neuropathic pain to be treated results from a soft-tissue injury.

9. The method of claim 8, wherein said soft-tissue injury is selected from the group consisting of pain associated with ligaments, tendons, muscles, bursa, sprains, strains, inflammations, contusions, arthritises, and post-surgical pains.

10. The method of claim 1 wherein said neuropathic pain is derived from one or more conditions selected from the group consisting of myofascial pains, fibromyalgia, bursitis, costrochondritis, repetitive motion injuries, carpal tunnel syndrome, and nociceptive pain.

11. A method for treating non-neuropathic pain comprising the step of:
topically administering a transdermal patch containing a pharmaceutical composition consisting of 5% lidocaine as an active ingredient, and the remainder consisting of inactive pharmaceutically acceptable materials.
